(19) United States
(12) Patent Application Publication
(10) Pub. No.: US 2020/0315856 A1
(43) Pub. Date: Oct. 8, 2020

(54) BANDAGE STRUCTURE

(71) Applicant: CHERNGER TECH. CO., LTD., Taipei City (TW)

(72) Inventor: MU-YI LIU, TAIPEI CITY (TW)

(21) Appl. No.: 16/838,683

(22) Filed: Apr. 2, 2020

(30) Foreign Application Priority Data
Apr. 3, 2019 (TW) 108112043

(51) Int. Cl. A61F 13/02

(52) U.S. Cl. CPC A61F 13/023; A61F 2013/00119; A61F 13/0246

(57) ABSTRACT

A bandage structure includes a substrate, an elastic sheet, a plurality of adhesive elements, and a plurality of protective layers. The substrate includes a hollow portion and a plurality of adhesive portions. The hollow portion includes a plurality of first elongated holes that are arranged in one row parallel to a length direction of the substrate. The adhesive portions are located on two sides of the hollow portion and the elastic sheet is located on the substrate. The elastic sheet includes an extension portion and a plurality of attaching portions. The extension portion corresponds in position to the hollow portion and has a plurality of second elongated holes. The second elongated holes are arranged in one row parallel to a length direction of the elastic sheet. The first elongated holes and the second elongated holes are staggered with each other along a length direction of the bandage structure.

### CROSS-REFERENCE TO RELATED PATENT APPLICATION

This application claims the benefit of priority to Taiwan Patent Application No. 108112043, filed on Apr. 3, 2019. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a bandage structure, and more particularly to a bandage structure for attaching to a wound instead of needles that is stitched at the wound, in which the bandage structure has a function of pulling a surrounding skin of the wound tightly together to accelerate a wound healing process.

### BACKGROUND OF THE DISCLOSURE

When there is a small wound on the human skin, a conventional medical bandage (such as an adhesive bandage) can cover the small wound to prevent infection to the small wound so that a healing process thereof can be accelerated. However, if there is a large wound on the human skin, a suture surgery would be needed to treat the large wound.

However, with respect to treatment of the small wound, while the conventional medical bandage covers the small wound, it cannot pull a surrounding skin of the small wound tightly together, so that a gap in the wound is formed, and the healing process thereof is decelerated. On the other hand, with respect to treatment of a larger wound by the suture surgery, anesthetics are needed to avoid pain during the suture surgery, and the anesthetics increase medical expenses. Furthermore, such treatment can leave scars that negatively affect aesthetical appearance.

### SUMMARY OF THE DISCLOSURE

In response to the above-referenced technical inadequacies, the present disclosure provides a bandage structure that is configured to quickly pull a surrounding skin of a large wound tightly together. When a person is injured in various places or environments, the bandage structure can be promptly attached to the wound to greatly reduce a time for the wound to heal, so that medical expenses can be saved.

In one aspect, the present disclosure provides the bandage structure including a substrate, an elastic sheet, a plurality of adhesive elements, and a plurality of protective layers. The substrate includes a hollow portion and a plurality of adhesive portions. The hollow portion includes a plurality of first elongated holes that are arranged in one row parallel to a length direction of the substrate. The adhesive portions are located on two sides of the hollow portion. The elastic sheet is located on the substrate and includes an extension portion and a plurality of attaching portions. The extension portion corresponds in position to the hollow portion and includes a plurality of second elongated holes that are arranged in one row parallel to a length direction of the elastic sheet. The first elongated holes and the second elongated holes are staggered with each other along the length direction of the bandage structure. The attaching portions are located on two sides of the extension portion. The adhesive elements are respectively located on the attaching portions, and the protective layers are detachably attached to the adhesive elements.

In certain embodiments of the present disclosure, a width of each of the attaching portions is less than a width of each of the adhesive portions.

In certain embodiments of the present disclosure, the first elongated holes and the second elongated holes do not overlap each other along the length direction of the bandage structure.

In another aspect, the present disclosure provides a bandage structure including an elastic sheet, a plurality of adhesive elements, and a plurality of protective layers. The elastic sheet includes an extension portion and a plurality of attaching portions. The extension portion includes a plurality of elongated holes, in which the elongated holes are arranged in one row parallel to a length direction of the elastic sheet. The attaching portions are located on two sides of the extension portion, and the adhesive elements are respectively located on the attaching portions. The protective layers are detachably attached to the adhesive elements.

In certain embodiments, the present disclosure provides a waterproof layer disposed on the elastic sheet.

In certain embodiments of the present disclosure, the waterproof layer is disposed on the extension portion instead of being disposed on the attaching portions.

Therefore, according to the certain embodiments of the present disclosure of the bandage structure, a user can stretch the elastic sheet toward two sides so that the adhesive elements have an appropriate distance between each other, and then the adhesive elements are attached to the skin at two sides of the wound. Accordingly, the skin at the two sides of the wound can be pulled tight in a direction of the wound through the elasticity of the elastic sheet, and then an area of the wound and the amount of tissue fluid that the cells need to secrete when the wound is healing are reduced. Accordingly, the wound healing process is accelerated. Furthermore, while the elongated holes can also serve as ventilation holes to allow the wound to breathe, the elongated holes still shield the wound as much as possible to avoid infection. Furthermore, the bandage structure can also be used with commercially available pharmaceuticals that have functions of sterilization, disinfection, and hemostasis.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective view of a bandage structure according to a first embodiment of the present disclosure.

FIG. 2 is an exploded view of the bandage structure according to the first embodiment of the present disclosure.

FIG. 3 is a cross-sectional view of the bandage structure according to the first embodiment of the present disclosure.

FIG. 4 is a schematic view of a first state of an elastic sheet of the bandage structure according to the first embodiment of the present disclosure.

FIG. 5 is a schematic view of second state of an elastic sheet of the bandage structure according to the first embodiment of the present disclosure.

FIG. 6 is a cross-sectional view of a bandage structure according to a second embodiment of the present disclosure.

FIG. 7 is a schematic view of a use state (a) of a bandage structure according to a third embodiment of the present disclosure.

FIG. 8 is a perspective view of the bandage structure according to the third embodiment of the present disclosure.

FIG. 9 is an exploded view of the bandage structure according to the third embodiment of the present disclosure.

FIG. 10 is a cross-sectional view of the bandage structure according to the third embodiment of the present disclosure.

FIG. 11 is a schematic view shows the bandage structure according to the third embodiment of the present disclosure attached to a wound.

FIG. 12 is a cross-sectional view of a bandage structure according to a fourth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

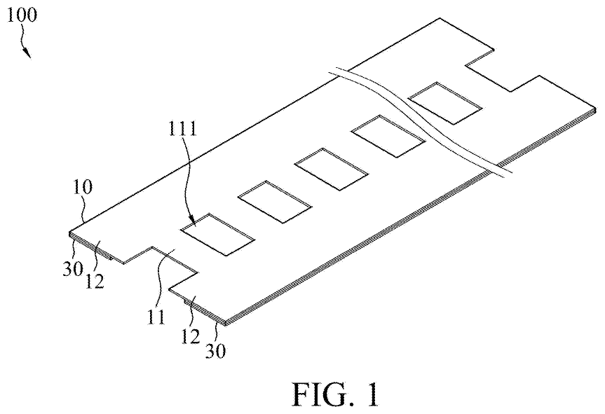

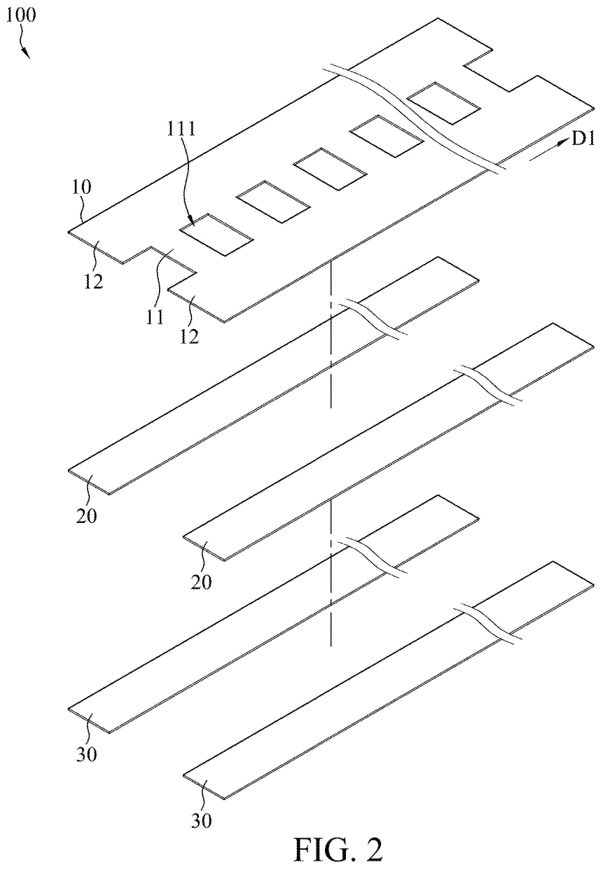

Reference is made to FIG. 1 to FIG. 3, which are respectively a perspective view, an exploded view, and a cross-sectional view of a bandage structure according to the first embodiment of the present disclosure. Referring to FIG. 1 to FIG. 3, the first embodiment of the present disclosure provides a bandage structure 100 including an elastic sheet 10, a plurality of adhesive elements 20, and a plurality of protective layers 30. In the present embodiment, the bandage structure 100 includes the two adhesive elements 20 and the two protective layers 30. The two adhesive elements 20 are attached to two sides of the elastic sheet 10, and the two protective layers 30 are attached to the two adhesive elements 20.

Referring to FIG. 1 to FIG. 3, the elastic sheet 10 includes an extension portion 11 and a plurality of attaching portions 12. In the present embodiment, the number of the attaching portions 12 is two, and the attaching portions 12 are located on two sides of the extension portion 11. The extension portion 11 includes a plurality of elongated holes 111 that are arranged in one row parallel to a length direction D1 of the elastic sheet 10. The elongated holes 111 are aligned with each other to provide the extension portion 11 with a ladder-like appearance. The extension portion 11 has elasticity and can be stretched toward two sides so that the two attaching portions 12 are distant relative to each other. After a stretching force of the extension portion 11 vanishes, the two attaching portions 12 shift to the extension portion 11. For example, the elastic sheet 10 can be made of elastic materials, such as elastic rubber materials, but it is not limited thereto. In other embodiments, it should be noted that the elastic sheet 10 can be configured such that only the extension portion 11 has elasticity, while the attaching portions 12 do not.

Referring to FIG. 1 to FIG. 3, the adhesive elements 20 are respectively located on the attaching portions 12. In the present embodiment, the adhesive element 20 is glue that is sticky and can be attached to a skin at two sides of the wound, so that the bandage structure 100 can be correspondingly fixed in position on the wound.

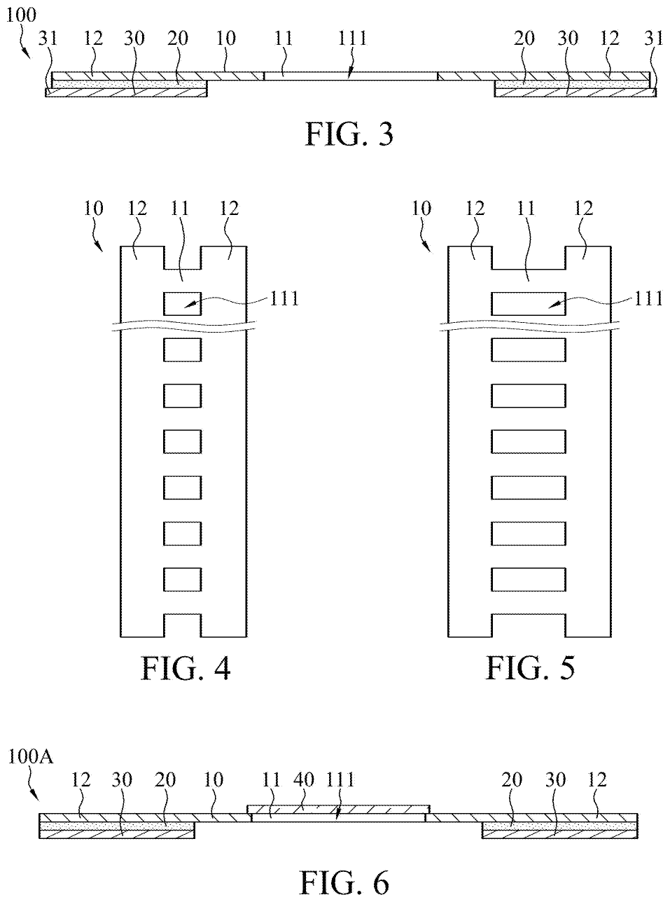

Referring to FIG. 1 to FIG. 3, the protective layers 30 are detachably attached to the adhesive elements 20. In a storage state (that is, a state that the bandage structure 100 is not used), the protective layers 30 is attached to the adhesive elements 20 so that the adhesive elements 20 on the same bandage structure 100 do not attach to each other and affect user operations. In a use state, the protective layers 30 can be removed (e.g., torn off) from the adhesive elements 20 so that the adhesive elements 20 can attach to the skin of an injured person. It should be noted that, in order to facilitate a user to remove the protective layers 30 from the adhesive elements 20, a length and a width of the protective layers 30 can each be slightly greater than the adhesive elements 20 to form protruding portions 31 (as shown in FIG. 3, the width of the protective layers 30 is slightly greater than the adhesive elements 20 to form the protruding portions 31). Therefore, the user can remove the protective layers 30 from the adhesive elements 20 through the protruding portions 31.

As mentioned above, since the elastic sheet 10 has elasticity, a distance between the two attaching portions 12 can be changed according to the state of the elastic sheet 10. Referring to FIG. 4 to FIG. 5, FIG. 4 is a schematic view of a first state of the elastic sheet of the bandage structure according to the first embodiment of the present disclosure. FIG. 5 is a schematic view of a second state of the elastic sheet of the bandage structure according to the first embodiment of the present disclosure. As shown in FIG. 4, when the extension portion 11 is in a loose state, a distance between the two attaching portions 12 is less. As shown in FIG. 5, when the extension portion 11 is in a stretched state, the distance between the two attaching portions 12 is greater.

According to the bandage structure 100 of one or more of the embodiments disclosed herein, the user can correspondingly attach the bandage structure 100 to the wound. The adhesive elements 20 are attached to the skin at the two sides of the wound. Specifically, the user can stretch the elastic sheet 10 toward two sides so that the attaching portions 12 are distant relative to each other, and then the user attaches the attaching portions 12 to the skin at the two sides of the wound through the adhesive elements 20. Accordingly, through the elasticity of the extension portion 11, the skin at the two sides of the wound can be pulled tight in a direction of the wound and then an area of the wound and the amount of tissue fluid that the cells need to secrete when the wound is healing are reduced. Accordingly, the wound healing process is accelerated without a suture surgery or pharmaceuticals. Furthermore, the elongated holes 111 of the extension portion 11 can be ventilation holes to allow the wound to breathe, but the elongated holes 111 still shield the wound as much as possible to avoid wound infection. Furthermore, the bandage structure 100 of the certain embodiments of the present disclosure can also be used with commercially available pharmaceuticals that have functions of sterilization, disinfection, and hemostasis. For example, the pharmaceuticals above can be applied to a medical alcohol cotton swab, and then covered on the wound together with the bandage structure 100. Alternatively, the wound can be sterilized with the pharmaceuticals before the bandage structure 100 is attached to the skin at the two sides of the wound.

### Second Embodiment

Reference is made to FIG. 6, which is a cross-sectional view of a bandage structure according to the second embodiment of the present disclosure. As shown in FIG. 6, in the present embodiment, the bandage structure 100A further includes a waterproof layer 40 that is disposed on the elastic sheet 10. In certain embodiments of the present disclosure, the waterproof layer 40 is disposed on the extension portion 11 instead of being disposed on the attaching portions 12. Accordingly, the bandage structure 100 has a waterproof effect and a dustproof effect for daily use and can provide convenience to the user in different situations.

### Third Embodiment

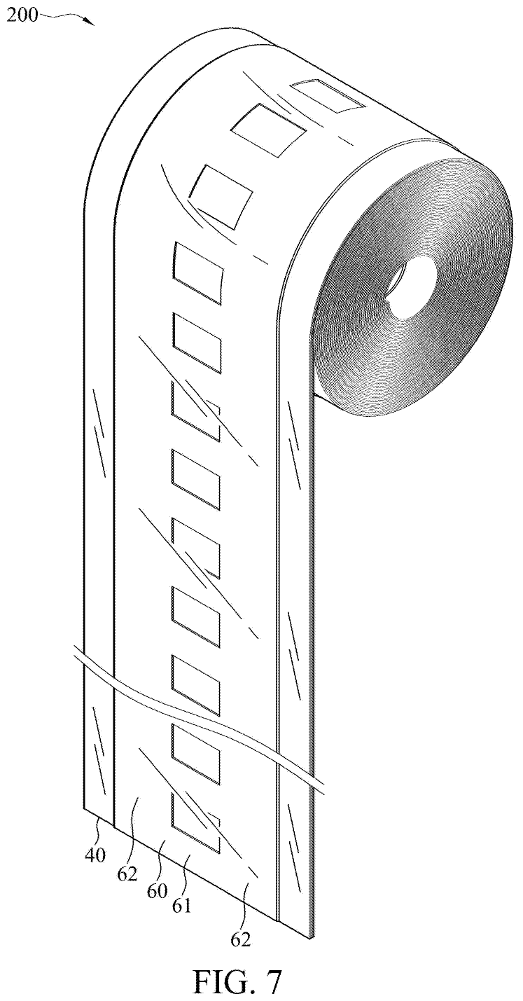

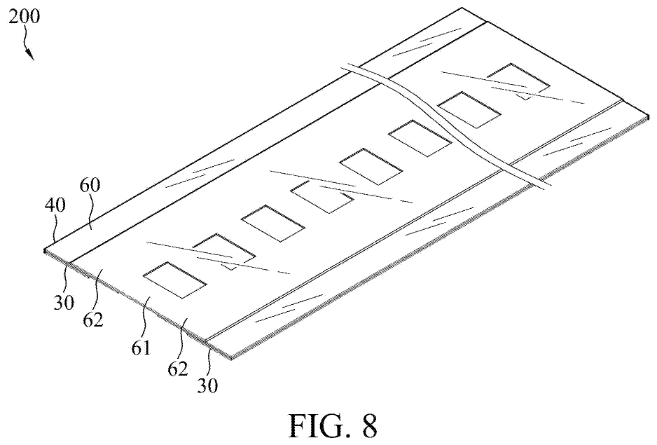

Reference is made to FIG. 7 to FIG. 10, which are respectively a schematic view, a perspective view, an exploded view, and a cross-sectional view of the bandage structure according to the third embodiment of the present disclosure. As shown in FIG. 7 to FIG. 10, a bandage structure 200 including a substrate 50, an elastic sheet 60, a plurality of adhesive elements 20, and a plurality of protective layers 30. As shown in FIG. 7, the bandage structure of the certain embodiments of the present disclosure can be made into a bandage roll for easy storage. The user can use a cutter to cut off an appropriate length of bandage from the bandage roll. As shown in FIG. 7 to FIG. 10, in the present embodiment, the bandage structure 200 also includes two adhesive elements 20 and two protective layers 30. The two adhesive elements 20 are attached to the two sides of the elastic sheet 10, and the two protective layers 30 are respectively attached to the two adhesive elements 20. The relevant technical details of the adhesive elements 20 and the protective layers 30 have already been described above, and will not be reiterated herein. The following description mainly focuses on the substrate 50 and the elastic sheet 60 of the third embodiment.

Referring to FIG. 7 to FIG. 10, the substrate 50 includes a hollow portion 51 and a plurality of adhesive portions 52. In the present embodiment, the number of the adhesive portions 52 is two, and the adhesive portions 52 are located on two sides of the hollow portion 51. The hollow portion 51 includes a plurality of first elongated holes 511 that are arranged in one row parallel to a length direction DO of the substrate 50. The first elongated holes 511 are aligned with each other to provide the hollow portion 51 with a ladder-like appearance. In certain embodiments, the substrate 50 basically does not have elasticity, so that when the user stretches the substrate 50 toward two sides using less force, two sides of the substrate 50 will not be distant from each other, but it is not limited thereto. In certain embodiments, the substrate 50 can have a slight elasticity so that when the user stretches the two sides of the substrate 50, the substrate 50 can stretch slightly to improve a practicality of the substrate 50.

Referring to FIG. 7 to FIG. 10, the elastic sheet 60 is located on the substrate 50. In the present embodiment, the elastic sheet 60 is adheringly disposed on the substrate 50, but it is not limited thereto. The elastic sheet 60 and the substrate 50 can also be connected to each other by other manners. The elastic sheet 60 includes an extension portion 61 and a plurality of attaching portions 62. In the present embodiment, the number of the attaching portions 62 is two, and the attaching portions 62 are located on two sides of the extension portion 61. The extension portion 61 corresponds in position to the hollow portion 51 and includes a plurality of second elongated holes 611. The second elongated holes 611 are arranged in one row parallel to a length direction D1 of the elastic sheet 60, so that the second elongated holes 611 are aligned with each other to provide the extension portion 61 with a ladder-like appearance. Furthermore, the first elongated holes 511 and the second elongated holes 611 are staggered with each other along the length direction of the bandage structure 200.

In the present embodiment, since the substrate 50 and the elastic sheet 60 extend along the length direction of the bandage structure 200, the length direction DO of the substrate 50 and the length direction D1 of the elastic sheet 60 are the same direction.

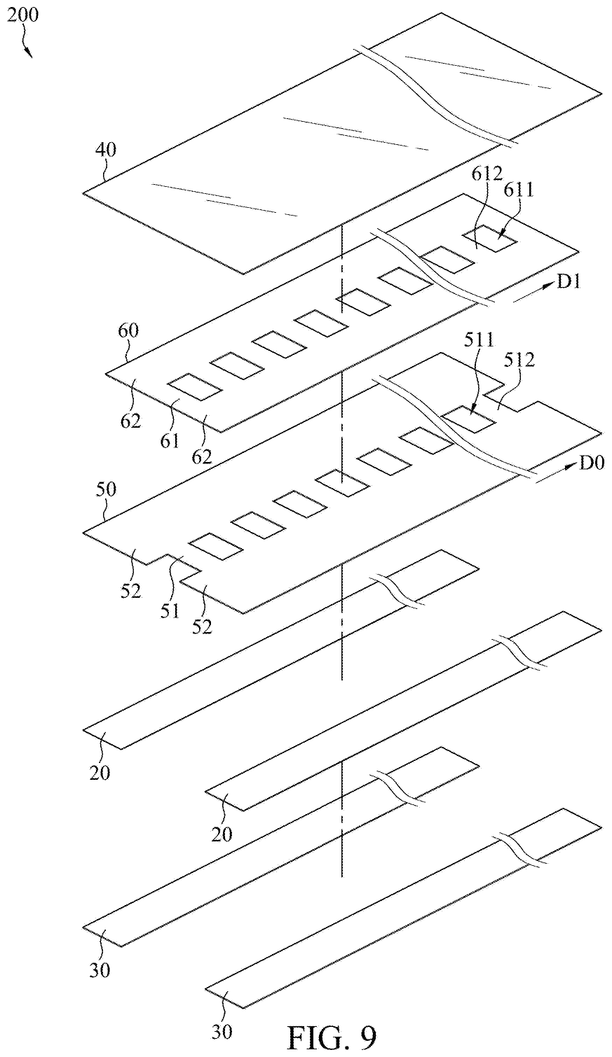

In certain embodiments, the first elongated holes 511 and the second elongated holes 611 do not overlap each other along the length direction of the bandage structure 200. As shown in FIG. 9, specifically, the hollow portion 51 of the substrate 50 includes a first connecting section 512 that is located between the first elongated holes 511 and the second elongated holes 611. The elastic sheet 60 includes the second elongated holes 611 and a second connecting section 612 that is located between the second elongated holes 511. The first connecting section 512 of the substrate 50 corresponds in position to the second elongated holes 611 of the elastic sheet 60, and the second connecting section 612 of the elastic sheet 60 corresponds in position to the first elongated holes 511 of the substrate 50. In other embodiments, a length of the first connecting section 512 of the substrate 50 can be different from the second elongated holes 611 of the elastic sheet 60. A length of the first elongated holes 511 of the substrate 50 can be different from the second connecting section 612 of the elastic sheet 60. That is, in these embodiments, the first elongated holes 511 and the second elongated holes 611 partially overlap (not shown).

Reference is made to FIG. 11, which is a schematic view showing the bandage structure according to the third embodiment of the present disclosure being attached to a wound site. As shown in FIG. 11, the adhesive elements 20 are respectively attached to the two sides of skin of the wound W so that the skin at two sides of the wound W close to the wound W (that is a center) and an area of the wound W is reduced. Accordingly, since the first connecting sections 512 of the substrate 50 do not have significant elasticity, the first connecting section 512 warps outward. Accordingly, since the second connecting section 612 of the elastic sheet 60 has elasticity, the second connecting section 612 is shortened and does not warp outward. Therefore, the second connecting section 612 that is shortened and the first connecting sections 512 that are warped outward correspond in position above the wound W.

According to the bandage structure 200 of the certain embodiments of the present disclosure, the user can correspondingly attach the bandage structure 200 to the wound. The adhesive elements 20 are attached to the skin at two sides of the wound. The first elongated holes 511 of the hollow portion 51 and the second elongated holes 611 are staggered with each other above the wound. Specifically, the user can stretch the bandage structure 200 toward two sides so that the two adhesive elements 20 are separated at an appropriate distance, and then the adhesive elements attach to the skin at two sides of the wound. Accordingly, through the elasticity of the elastic sheet 60, the skin at two sides of the wound can be pulled tight in a direction of the wound through the elasticity of the elastic sheet, and then an area of the wound and the amount of tissue fluid that the cells need to secrete when the wound is healing are reduced. Accordingly, the wound healing process is accelerated without a suture surgery or pharmaceuticals. Furthermore, the first elongated hole 511 of the hollow portion 51 and the second elongated hole 611 of the extension portion 61 can be a ventilation hole to allow the wound to breathe, but still shield the wound as much as possible to avoid infection. Furthermore, the bandage structure 200 of the certain embodiments of the present disclosure can also be used with commercially available pharmaceuticals that have functions of sterilization, disinfection, and hemostasis. For example, the pharmaceuticals above can be applied to a medical alcohol cotton swab, and then covered on the wound together with the bandage structure 200. Alternatively, the wound can be sterilized with the pharmaceuticals before the bandage structure 100 is attached to the skin at the two sides of the wound.

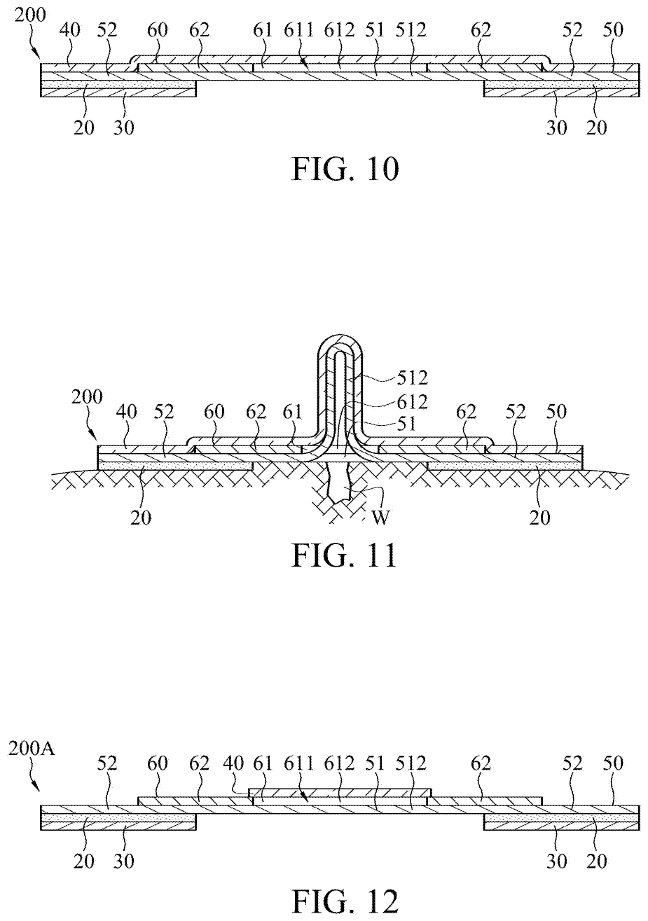

In certain embodiments, as shown in FIG. 9 and FIG. 10, a width of the attaching portions 62 is less than a width of the adhesive portions 52 corresponding in position to the attaching portions 62, but the attaching portions 62 and the adhesive portions 52 can still be fixed to each other. Accordingly, the amount of material required for manufacturing the bandage structure 200 can be reduced, so that the material cost can be saved.

As shown in FIG. 7 to FIG. 10, in certain embodiments of the present disclosure, a bandage structure 200A further includes a waterproof layer 40 that is disposed on the elastic sheet 60. Furthermore, in some embodiments, as shown in FIG. 12, the waterproof layer 40 is disposed on the extension portion 61 instead of being disposed on the attaching portion 62. Accordingly, the bandage structure 200A has a waterproof effect and a dustproof effect for daily use and can provide convenience to the user in different situations.

In certain embodiments, the elongated holes 111, the first elongated holes 511, and the second elongated holes 611 can be rectangular holes, but it is not limited thereto. In some embodiments, the elongated holes 111, the first elongated holes 511, and the second elongated holes 611 can also be oblong holes or rectangular holes with slightly rounded corners.

In certain embodiments, the substrate 50 can be a plastic sheet and have a width of 38 mm The waterproof layers 40 and the protective layers 30 can be PVC plastic sheets. The adhesive portions 52 of the substrate 50 can each have a width of 15 mm, and the hollow portion 51 can have a width of 8 mm Furthermore, a distance between the first elongated hole 511 and the second elongated holes 611 can be 5 mm, and the thickness of the substrate 50, the elastic sheet 60, and the adhesive elements 20 can be within a range of 0.03 mm to 0.1 mm It should be noted that the above dimensional design is for illustrative purposes only, and the present disclosure is not limited thereto.

In conclusion, according to the certain embodiments of the present disclosure of the bandage structure, a user can stretch the elastic sheet toward two sides so that the adhesive elements have an appropriate distance between each other, and then the adhesive elements are attached to a skin at two sides of the wound. Accordingly, the skin at the two sides of the wound can be pulled tight in a direction of the wound through the elasticity of the elastic sheet, and then an area of the wound and the amount of tissue fluid that the cells need to secrete when the wound is healing are reduced. Accordingly, the wound healing process is accelerated. Furthermore, the elongated holes can also serve as ventilation holes to allow the wound to breathe, the elongated holes still shield the wound as much as possible to avoid infection. Furthermore, the bandage structure can also be used with commercially available pharmaceuticals that have functions of sterilization, disinfection, and hemostasis.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A bandage structure, comprising:
an elastic sheet including:
an extension portion including a plurality of elongated holes that are arranged in one row parallel to a length direction of the elastic sheet; and
a plurality of attaching portions located on two sides of the extension portion;
a plurality of adhesive elements respectively located on the attaching portions; and
a plurality of protective layers detachably attached to the adhesive elements.

2. The bandage structure according to claim 1, further comprising a waterproof layer disposed on the elastic sheet.

3. The bandage structure according to claim 2, wherein the waterproof layer is disposed on the extension portion.

4. A bandage structure, comprising:
a substrate including:
a hollow portion including a plurality of first elongated holes that are arranged in one row parallel to a length direction of the substrate; and
a plurality of adhesive portions located on two sides of the hollow portion;
an elastic sheet located on the substrate and including:
an extension portion corresponding in position to the hollow portion and including a plurality of second elongated holes, wherein the second elongated holes are arranged in one row parallel to a length direction of the elastic sheet, and wherein the first elongated holes and the second elongated holes are staggered with each other along a length direction of the bandage structure; and
a plurality of attaching portions located on two sides of the extension portion, wherein the attaching portions are respectively located on the adhesive portions;
a plurality of adhesive elements located on the attaching portions; and
a plurality of protective layers detachably attached to the adhesive elements.

5. The bandage structure according to claim 4, wherein a width of each of the attaching portions is less than a width of each of the adhesive portions.

6. The bandage structure according to claim 4, further comprising a waterproof layer disposed on the elastic sheet.

7. The bandage structure according to claim 6, wherein the waterproof layer is disposed on the extension portion.

8. The bandage structure according to claim 4, wherein the first elongated holes and the second elongated holes do not overlap each other along the length direction of the bandage structure.
